(19) United States
(12) Patent Application Publication
(10) Pub. No.: US 2020/0041430 A1
(43) Pub. Date: Feb. 6, 2020

(54) DEVICES AND METHODS TO EVALUATE TISSUE COOLING

(71) Applicant: Wake Forest University Health Sciences, Winston-Salem, NC (US)

(72) Inventors: F. Scott Gayzik, Winston-Salem, NC (US); Ryan D.M. Packett, Winston-Salem, NC (US); Philip J. Brown, Winston-Salem, NC (US); Gautam Popli, Lewisville, NC (US); Megan Fritz, Naperville, IL (US)

(21) Appl. No.: 16/339,789

(22) PCT Filed: Oct. 6, 2017
(86) PCT No.: PCT/US17/55520

§ 371 Date: Apr. 5, 2019

Related U.S. Application Data
(60) Provisional application No. 62/405,046, filed on Oct. 6, 2016.

(51) Int. Cl. G01N 25/00

(52) U.S. Cl. CPC G01N 25/00; A61F 7/0085

(57) ABSTRACT

The present invention provides devices, systems, and methods for using them to monitor therapeutic cooling of blood perfused tissues or organs such as the brain. In one embodiment, the invention comprises a device for evaluating tissue cooling comprising a container that contains a polymer-fluid matrix having a thermal cooling property that is substantially similar to a tissue or organ such as a human brain. In another embodiment, the invention comprises a system for evaluating tissue cooling comprising: (a) a device for evaluating tissue cooling as described above and (b) a warm loop. In another embodiment, the invention comprises a method for evaluating a technique for cooling a tissue or organ comprising (a) providing a device comprising a container that contains a polymer-fluid matrix having a thermal cooling property that is substantially similar to a tissue or organ such as human brain, and (b) perfusing the polymer-fluid matrix with a fluid.

### CROSS-REFERENCE TO RELATED APPLICATIONS

This application claims priority to U.S. Provisional Patent Application No. 62/405,046 entitled “DEVICES AND METHODS TO EVALUATE TISSUE COOLING,” filed Oct. 6, 2016, the entirety of which is hereby incorporated by reference.

### FIELD OF THE INVENTION

This invention relates to evaluating therapeutic tissue cooling devices, techniques, and methods.

### BACKGROUND

Clinically induced hypothermia has been explored for some time now as an effective treatment for various injuries and ailments. Tissue cooling has been proven as a viable therapy for multiple conditions and injuries, as applied to various organs and tissues. For example, tissue cooling can be applied to the head to treat epilepsy and concussions, leading to improved long-term outcomes. In the case of the brain, it can be used to treat such conditions as epilepsy and traumatic brain injury (TBI) by slowing down electrophysiological and metabolic activity.

Devices for cooling portions of the brain and other blood perfused tissues and organs continue to be developed. Some research has been done on determining how much cooling is required to provide the desired therapeutic effect, however, very few reliable methods exist for predicting how much cooling certain technologies can provide. Clinical trials are expensive and risky and mathematical modeling is limited in its ability to capture the precise details of unique designs.

Thus, to better understand how much temperature reduction can be achieved by different cooling methods, it would be helpful to model the interaction between tissue cooling therapy and blood perfused organs or tissues, such as the human brain. There is therefore a need in the scientific and clinical community to develop a model which can simulate the cooling effects of various devices on the brain and other blood perfused organs or tissues.

### SUMMARY

The present invention provides devices and systems, and methods of use of such devices and systems to monitor therapeutic cooling of blood perfused organs and tissues such as the brain.

In one embodiment, the invention comprises a device for evaluating tissue cooling comprising a container that contains a polymer-fluid matrix having a thermal cooling property that is substantially similar to a blood perfused human tissue or organ such as brain. In some embodiments, the polymer is a super absorbent polymer (SAP) that, when hydrated, has thermal properties similar to water. In some embodiments, the fluid is water. In some embodiments, the tissue is a blood perfused organ such as the brain. In some embodiments, the thermal cooling property for at least a portion of the polymer-fluid matrix contained therein has a thermal property that deviates less than about 2° C. from that of normal tissue, such as brain tissue, for a time that is substantially similar to the time used to cool a tissue or organ, such as human brain.

In another embodiment, the invention comprises a system for evaluating tissue cooling, such as brain cooling, comprising: (a) a container comprising a polymer-fluid matrix having a thermal cooling property that is substantially similar to a human organ such as brain; and (b) a warm loop. In some embodiments, the warm loop contains a fluid having a temperature of about physiological temperature when passing through the inlet of the container. In some embodiments, the perfusion of flow ranges from about 30,000 to about 35,000 W/m³C. In some embodiments, flow rate within the warm loop ranges from about 2 to about 7 gph.

In yet another embodiment, the invention comprises a method for evaluating a technique for cooling a blood perfused tissue or organ such as brain comprising (a) providing a device comprising a container that contains a polymer-fluid matrix having a thermal cooling property that is substantially similar to a tissue or organ such as human brain, and (b) perfusing the polymer-fluid matrix with a fluid. In some embodiments, perfusing the polymer-fluid matrix with a fluid maintains the temperature of a portion or all the polymer-fluid matrix at about physiological temperature. In some embodiments, the method further comprises measuring the temperature of the polymer-fluid matrix at various depths in the matrix. In some embodiments, the method further comprises cooling the polymer-fluid matrix with a cold loop. In some embodiments, the method further comprises comparing the measured temperatures to a modeled condition for thermal cooling.

### BRIEF DESCRIPTION OF THE FIGURES

The present invention may be better understood by referring to the following non-limiting figures. The figures are intended to illustrate certain embodiments and/or features of the invention, and to supplement any description(s) of the invention. The figures do not limit the scope of the invention, unless the written description expressly indicates that such is the case.

FIG. 1 shows a front view of an embodiment of the device. Inlet and outlet ports permit flow of a fluid through a container which contains a polymer-fluid matrix.

FIG. 2 shows a front view of an embodiment of the device further equipped with a cooling device attached to a fixture. The fixture ensures consistent placement of the cooling device on the polymer-fluid matrix during each use.

FIG. 3 is a diagram depicting an embodiment of the system for evaluating tissue or organ cooling (e.g., brain cooling). A warm loop delivers fluid to the container which contains the polymer-fluid matrix, maintaining the matrix at a desired temperature (e.g. physiological temperature). A cold loop delivers fluid to the cooling device, which cools at least a portion of the polymer-fluid matrix.

FIG. 4 is a graph comparing a theoretical model to experimentally measured temperatures of the polymer-fluid matrix in a transient temperature response experiment using an embodiment of the system. After steady state temperature was achieved, the polymer-fluid matrix was cooled using an approximately 2° C. cooling device, and temperature data was recorded at a frequency of 1 Hz and a depth of 5 mm. Data were also collected and modeled for the recovery phase in which the cooling device was removed and the temperature was permitted to increase.

FIG. 5 is a graph comparing a theoretical model to experimentally measured temperatures at varying depths within the polymer-fluid matrix in transient temperature response experiments using an embodiment of the system. Data were collected as described for FIG. 4, with the exception that data for the recovery phase was not collected. Data were recorded and modeled at depths of 5 mm, 10 mm, and 15 mm.

FIG. 6 shows the temperature profile effects over time at each of the different depths (surface, 5 mm, 10 mm, 15 mm) in an embodiment of the brain phantom with a perfusion rate of 4 gallons per hour. Other rates were tested, and 4 gallons per hour corresponded to the most physiologically accurate perfusion rate.

FIG. 7 shows comparisons of steady state temperatures achieved at 5 mm depth in and embodiment of the brain phantom for different perfusion rates, as predicted by mathematical model (“model”) and as experimentally observed (“experiment”). At 5 mm depth, the brain phantom demonstrates an appropriate qualitative response to increasing perfusion rate: as the perfusion rate of warm blood simulant increases so does the steady state temperature under the same cooling conditions.

FIG. 8 shows the same comparisons as FIG. 7 but for 10 mm depth in an embodiment of the brain phantom. The trend of increasing steady state temperatures is observed, but the experimental observations were not as close to the values predicted by the model for 10 mm depth as for 5 mm.

### DETAILED DESCRIPTION

Titles or subtitles may be used in the specification for the convenience of a reader, which are not intended to influence the scope of the present invention.

It is to be understood, although not always explicitly stated, that the reagents described herein are merely exemplary and that equivalents of such are known in the art.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Known methods and techniques are generally performed according to conventional methods well known in the art and as described in various general and more specific references that are discussed throughout the present specification unless otherwise indicated. The nomenclatures used in connection with the medical and/or surgical procedures and techniques described herein are those well-known and commonly used in the art.

The following terms, unless otherwise indicated, shall be understood to have the following meanings:

As used herein, the terms “a,” “an,” and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a range of 1-3 refers to ranges having 1, 2, or 3. Similarly, a range of 1-5 refers to ranges having 1, 2, 3, 4, or 5, and so forth.

Throughout this application, the term “about” is used to indicate that a value includes the inherent variation of error for the device, the method being employed to determine the value, or the variation that exists among samples. It is to be understood, although not always explicitly stated, that all numerical designations may be preceded by the term “about.”

The term “assess” or “assessing” means to evaluate, determine, or grade, particularly in reference to a parameter. Thus, to assess means to assign a value or a grade to an inquiry, particularly a parameter which can be tested and observed. Assessing a parameter may be qualitative or quantitative, and may also be expressed as a qualitative or quantitative range.

The term “comprising” or “comprises” is intended to mean that the devices, systems and methods include the recited elements, but not excluding others. “Consisting essentially of” when used to define devices, systems and methods, shall mean excluding other elements of any essential significance to the combination. For example, a device consisting essentially of the elements as defined herein would not exclude other elements that do not materially affect the basic and novel characteristic(s) of the claimed invention. “Consisting of” shall mean excluding essential elements and substantial method steps that are not included. Embodiments defined by each of these transition terms are within the scope of this invention.

As used herein, “concurrent” or “concurrently” refers to performing at least two steps at the same time or at approximately the same time. The term is also intended to included instances in which a first step is initiated before a second step, but in which the second step initiates while the first step continues to proceed such that the first step and the second step occur at the same time during at least a portion of the duration of the first step.

The term “convection” refers to a form of movement of molecules within fluids. As used herein, convection refers to the circulation of molecules due to unequal heat distribution between molecules. Warmer, less densely packed molecules tend to rise in fluid whereas cooler, more densely packed molecules tend to settle. One result of such circulating fluids is heat transfer between circulating molecules.

The term “evaluate” is similar to the term “assess” and in some instance can have the same meaning. To evaluate means to assign value. To evaluate can mean to undertake the process of determining assignable values or can mean interpreting assignable values. For instance, a tissue cooling system may be evaluated experimentally by performing the herein described methods, or may be evaluated by interpreting data obtained by performing the herein described methods. Evaluation may be qualitative or quantitative, and may also be expressed as a qualitative or quantitative range. As examples, a tissue cooling device may be good or bad, useful or not useful, effective or ineffective, etc., or may be 10% more effective than a standard, or may be approximately 10-20% more effective than a standard. As used herein, evaluation is based on observation of a device, method, system, theory, model, etc. in use, experiments, models, etc., or on observation of data, information, feedback, etc. gained therefrom. The actual act of assigning value may be performed by a separate entity, e.g. human or computer, than the entity obtaining the data, information, feedback, etc.

The term “loop” refers to any theoretical or structural arrangement or process in which the end returns to the beginning. The end can, but need not, proceed to another partial or full iteration from the beginning (in other words, repeat). Typically, the loop as used herein refers to a fluid loop. Fluid may begin from an arbitrary starting point and flow through the loop towards an arbitrary ending point. The fluid loop may continue to proceed such that molecules pass through the entirety of the loop one or more times. A loop often is substantially enclosed to compartmentalize the fluid flowing through the loop. However, portions of the loop can be open. For example, a warm loop delivers and receives fluid to/from a container which may be open. However, the open container (or any open portion of the loop) should substantially contain the fluid, preventing escape or loss of the fluid from the loop. A loop may contain many additional components which influence the properties of the loop. For example, the loop can contain heating and/or cooling devices to modulate loop thermodynamics, or pumps to modulate loop fluid dynamics.

The term “matrix” refers to a structural medium. The matrices used herein are comprised of a polymer and a fluid. The term matrix excludes solid structures which are, as a whole, completely and fully impenetrable or impervious to fluids. A matrix of the present invention requires a means for fluid to permeate or flow through, between, or within the solid formations of the matrix.

The term “model,” “modeled,” “modeling,” or “modeled condition” refers to forming a representation, imitation, or resemblance. The model may be tangible, e.g. a matrix which models the properties of a tissue or organ such as brain, or conceptual, e.g. a calculation which models the properties of a tissue or organ such as brain. A model need not, and frequently cannot, be an exact replica of the thing to be modeled. A model provides a means to gather information about the model which is relevant to thing being modeled. Often, the information obtained from a model is used to make predictions about the thing being modeled. A “modeled condition” is a species of the genus term “model” which refers to a specific condition or set of conditions which are modeled. In a modeled condition, it is irrelevant if another condition which is not a member of the modeled condition is significantly different or contains little or no representation, imitation, or resemblance to that which is being modeled.

“Optional” or “optionally” means that the subsequently described event or circumstance can or cannot occur, and that the description includes instances where the event or circumstance occurs and instances where it does not.

The term “perfusion” refers to the flow of fluid over or through non-fluids. In biological subjects and samples therefrom, perfusion refers to passage of fluid, e.g. blood, through vessels, e.g. veins, arteries, and capillaries. Thus, perfusion distributes fluid throughout the biological subject or sample therefrom. In a matrix, perfusion is the flow of fluid over, between, around, or through the matrix, in particular the particles of the matrix.

The term “physiological temperature” refers to the average temperature or range of temperatures for a given animal. Physiological temperatures differ between animals, for instance between mammals and amphibians. Further, any two individuals of the same species can have different measured physiological temperatures. As such, physiological temperature is best expressed as a range typical for the species. Average physiological temperatures are known for many common species of animals. For humans, the physiological temperature typically ranges from about 36.1° C. to about 37.5° C., or depending on more stringent criteria, can range from 36.5° C. to about 37.2° C. For simplicity and without limiting scope, the average physiological temperature for humans is sometimes reported as about 37° C.

The term “subject” and “individual” means any animal, particularly any mammal, possessing blood perfused tissues and organs, such as a brain, brain tissue, or other biological samples for which cooling techniques may be evaluated on. The subject may be, for instance, a horse, cow, pig, monkey, rodent such as rat, mouse, or hamster, or can be human. The term encompasses any animal in any state, for instance alive, dead, unconscious, unresponsive, sedated, etc.

The term “substantially” refers to all, nearly all, or, in some instances, mostly all of a thing, trait, property, action, etc. to which the term modifies. The term includes situations in which the comparative entity does not contain all of a thing, trait, property, action, etc., but does contain a sufficient amount of said thing, trait, property, action, etc. that it may be considered similar enough, for practical purposes under the circumstances, to be thought of as having all of the relevant thing, trait, property, action, etc. Thus, minor deviations which distinguish the comparative entity but do not amount to a substantive difference are inclusive in the term. The term excludes situations in the differences in a comparative entity's thing, trait, property, action, etc. are sufficiently large enough that the comparative entity would be considered wholly and substantively distinctive or distinguishable, such that the comparison is of little value. For example, a matrix having cooling properties substantially similar to a tissue or organ such as brain must be a matrix that, for all practical purposes under the circumstances of modeling cooling of tissue using a polymer-fluid matrix, has cooling properties that are similar enough to a tissue or organ such as brain that the matrix may be thought of as having all of the relevant cooling properties of that tissue or organ. However, a matrix will likely have some differences in cooling properties as compared to a tissue or organ such as brain, but these differences cannot result in the cooling properties of a matrix being wholly and substantively distinctive or distinguishable from the tissue or organ such that the comparison between cooling properties of a matrix and cooling properties of the tissue or organ are of little value.

The term “thermal property” refers to any property or trait in relation to thermodynamics. The thermal property of a tissue or organ, e.g. a brain, can be, among other things, the present or changing temperature, the tendency to lose or gain heat, the thermal-modulated properties (e.g. fluidity, surface tension, or density of a fluid), and/or other properties. Frequently, the thermal property refers to temperature dynamics over a period of time. It is to be understood that multiple or even infinite portions of a single entity, e.g. a brain, can have different thermal properties at the same time. For instance, brain tissue 5 mm below a contacting cooling device may have different thermal properties (e.g. changes in temperature over time) than brain tissue of the same brain but which is 10 mm below the cooling device.

The term “thermal cooling property” is a species of the genus term “thermal property.” The term specifically refers to the decrease in temperature over time. The term typically refers to decreases in temperature in response to a stimulus, e.g. a cooling device. It should be understood that the term encompasses actual increases in temperature, but which are expressed as negative values.

The terms “user” and “operator” are used interchangeably to refer to a person who operates a device and/or system of the invention.

### Devices for Evaluating Tissue Cooling

In certain aspects, the present invention utilizes the thermal properties of a polymer-fluid matrix which resemble the thermal properties of a human tissue or organ such as brain. A container contains the polymer-fluid matrix and is optionally equipped with an inlet and an outlet to circulate fluid. Circulating fluid adjusts and/or maintains the temperature of a portion or all the polymer-fluid matrix. Because of the unique design of the device and the arrangement and thermal properties of the polymer-fluid matrix, cooling the polymer-fluid matrix in the device models the cooling of a tissue or organ such as brain or brain tissue.

In certain aspects, the invention comprises a device for evaluating tissue or organ cooling comprising a container that contains a polymer-fluid matrix having a thermal cooling property that is substantially similar to a tissue or organ such as human brain. In some embodiments, the polymer is a super absorbent polymer that, when hydrated, has thermal properties similar to water. In some embodiments, the fluid is water. In some embodiments, the thermal cooling property for at least a portion of the polymer-fluid matrix contained therein has a thermal property that deviates less than about 2° C. from that of normal tissue, such as normal brain tissue, for a time that is substantially similar to the time used to cool a tissue or organ such as a human brain.

The device includes a container that contains a polymer-fluid matrix. The container provides a housing for the polymer-fluid matrix and for optional fluid which may permeate the polymer-fluid matrix. The container may be formed from any material which can contain fluids and withstand temperature fluctuations at least between about physiological temperature and about 25° C., more preferably between about physiological temperature and 0° C., and more preferably between about 10° C. above physiological temperature to about −10° C. Examples of suitable materials for the container include, without limitation, plastics, rubber, polymers such as acrylic, glass, metals including polymer-coated metals, foam such as Styrofoam, and the like. The container may optionally be coated, for instance, to prevent fluid leakage and/or avoid undesirable interactions between the container material and the circulating fluid. An insulating layer may optionally be applied to the container. An insulating layer can be wrapped, adhered, sprayed, and/or affixed to the container, and can further maintain temperature by reducing thermal escape. The top of the container can be open, partially covered, or fully covered. Alternatively, the top of the container can be open to receive a cooling device and subsequently covered to seal the cooling device within the container.

The container optionally includes an inlet for pumping fluid into the container, and optionally includes an outlet for removing fluid from the container. In some embodiments, the container includes more than one inlet and more than one outlet. Each inlet can be, but need not be, paired with an outlet. Thus, there can be a different number of inlets than outlets. Fluids flowing into the container through separate outlets can be the same or different fluids which mix within the container. Inlets and outlets can be located at varying positions on the container. In some embodiments, the inlets can be positioned in the upper half of the container and the outlets can be positioned in lower half of the container, or alternatively, this arrangement can be reversed. In some embodiments, an outlet and inlet pair are positioned on each side surface of the container. As an example, a cubic container has four vertical sides in which 4 pairs of inlets and outlets may be attached. In some embodiments, the more than one inlet, the more than one outlet, and/or the more than one inlet and more than one outlet are of different sizes, e.g. different diameters. Along with other parameters such as pressure and flow rate, inlet and outlet size affects flow dynamics. The device includes a polymer-fluid matrix contained within the container. The matrix is comprised of a polymer and a fluid. Blood perfused tissues and organs (such as brain), and blood itself, are comprised of primarily water and, as such, have thermal properties reflective of the high amounts of water contained therein. Thus, the polymer optionally can be a super absorbent polymer having thermal properties similar to water. Alternatively, the polymer can have thermal properties similar to water when hydrated. Thus, a super absorbent polymer can optionally be hydrated with the fluid to form a polymer-fluid matrix having thermal properties similar to water.

The polymer can be provided in various forms. Non-limiting examples in which the polymer can be provided include gels, powder, beads, particles, fibers, pellets, crystals, slurry, aqueous solution, and the like. In some embodiments, the polymer is provided in varying sizes of one or more forms. As an example, the polymer can be provided as particles having a range of sizes. For instance, the sizes may range from about 10 mm in diameter to microscopic diameters. As used herein, the term “microscopic” means discernible only under the aid of the magnifying power of a microscope but not discernible to the average human eye alone. In some embodiments, the particle sizes may range from about 100 μm to about 4 mm. For example, particle sizes may include Small Granulation (Type S) of 100-800 μm, Medium Granulation (Type M) of 800-1500 μm, Large Granulation (Type L) of 2-4 mm, and SAP spheres of 1 mm, 2 mm, 4 mm, or other sizes. In some embodiments, a mixture of two or more particle sizes can be used.

The polymer can be any polymer which, when in a polymer-fluid matrix, contains thermal properties similar to biological tissues, e.g. brain tissue and/or blood. Non-limiting examples include polymers of carboxylic acids, unsaturated carboxylic acids, vinyl carboxylic acids, amides, and vinyl amides. In some embodiments, the polymer can be an acrylic acid polymer. In some embodiments, the polymer can be an acrylamide polymer. In some embodiments, the polymer can be an acrylic acid-acrylamide co-polymer. In some embodiments, the polymer can be potassium neutralized. Other hydrogels and hydrogel suspensions may also be appropriate.

The fluid of a polymer-fluid matrix can be any fluid which results in a polymer-fluid matrix that contains thermal properties similar to biological tissues, e.g. brain tissue and/or blood. Non-limiting examples of fluids include water, saline, ethanol, ethylene glycol, dimethyl sulfoxide, acetic acid, acetonitrile, ethyl acetate, isopropanol, butanol, methanol, and bodily fluids such as plasma, blood, interstitial fluid, or lymph, and mixtures thereof. In a preferred embodiment, the fluid is water.

In some embodiments, a fluid is added to a solid form of the polymer, e.g. particles, to hydrate the polymer. Optionally, the polymer absorbs the fluid and swells, resulting in a polymer-fluid matrix. As used herein, the term “swell” or “swells” refers to an expansion or increase in size of the polymer, e.g. the polymer increases in displacement volume. In some embodiments, the polymer swells to at least about 10, at least about 50, at least about 100, at least about 200, or at least about 300 times its original size or original displacement volume. In some embodiments, the hydrated polymer-fluid matrix contains at least about 50%, at least about 75%, at least about 90%, at least about 95%, at least about 98%, or at least about 99% fluid by volume. Swelling of the polymer to contain high levels of fluid at least partially imparts thermal properties on the polymer-fluid matrix which are similar to water and to predominantly water-containing tissue and blood.

The polymer-fluid matrix can be characterized by a variety of properties. To effectively evaluate therapeutic tissue cooling devices, techniques, and methods, the matrix may be designed to resemble the properties of the target tissue or organ (e.g., brain tissue) and/or blood. Brain tissue typically has a density of about 1040 kg/m³, while blood typically has a slightly higher density of about 1050 kg/m′. However, pure water has a density of about 1000 kg/m³. The herein described polymer-fluid matrix can optionally have a density from about 500 to about 2,000 kg/m³, from about 700 to about 1800 kg/m³, from about 800 to about 1500 kg/m³, from about 900 to about 1200 kg/m³, from about 950 to about 1,100 kg/m³, from about 980 to about 1075 kg/m³, or from about 990 to about 1050 kg/m³. In some embodiments, the polymer-fluid matrix can optionally have a density from about 995 to about 1005 kg/m³, or about 1000 kg/m³. In some embodiments, the polymer-fluid matrix can optionally have a density from about 1035 to about 1055 kg/m³, or about 1040 kg/m³, or about 1050 kg/m³. Brain tissue typically has a thermal conductivity of about 0.5 watts per meter*kelvin (W/mK), while blood typically has a slightly higher thermal conductivity of about 0.52 W/mK. However, pure water has a thermal conductivity of about 0.63 W/mK. The herein described polymer-fluid matrix can optionally have a thermal conductivity from about 0.1 to about 1.5 W/mK, from about 0.2 to about 1.2 W/mK, from about 0.3 to about 1.0 W/mK, from about 0.4 to about 0.7 W/mK, or from about 0.45 to about 0.65 W/mK. In some embodiments, the polymer-fluid matrix can optionally have a thermal conductivity from about 0.48 to about 0.54 W/mK, or about 0.5 W/mK, or about 0.52 W/mK. In some embodiments, the polymer-fluid matrix can optionally have a thermal conductivity from about 0.6 to about 0.65 W/mK, or about 0.63 W/mK. Brain tissue typically has a specific heat of about 3560 joules per mole*kelvin (J/molK), while blood typically has a slightly higher specific heat of about 3617 J/molK. However, pure water has a specific heat of about 4178 J/molK. The herein described polymer-fluid matrix can optionally have a thermal conductivity from about 2000 to about 5000 J/molK, from about 2500 to about 4500 J/molK, from about 3000 to about 4300 J/molK, from about 3400 to about 4250 J/molK, or from about 3500 to about 4200 J/molK. In some embodiments, the polymer-fluid matrix can optionally have a specific heat from about 3550 to about 3625 J/molK, or about 3560 J/molK, or about 3617 J/molK. In some embodiments, the polymer-fluid matrix can optionally have a specific heat from about 4170 to about 4190 J/molK, or about 4178 J/molK.

Tissues and organs (such as brain) are perfused with blood vessels and capillaries which compartmentalize hemostatic blood circulation. The herein described matrix can optionally contain fluid-swollen particles having space between the particles. Fluid can flow, or perfuse, through the spaces between particles, thus compartmentalizing fluid flow. Thus, fluid flow dynamics within the polymer-fluid matrix may optionally model hemostatic blood circulation. Fluid flow dynamics in the matrix may be altered by a range of parameters understood by one of skill in the art. As non-limiting examples, larger spacing between particles reduces flow rate and pressure, increased perfusion may decrease pressure, and increased solute concentration may increase pressure. In some embodiments, the polymer can contain two or more sizes of particles. In some embodiments, two or more sizes of particles can be arranged in layers. Optionally, layering of the particles can be ordered from largest to smallest particles. Optionally, the top layer may contain the smallest particles. Such an arrangement can position the smallest particles at the uppermost region of the matrix and exposed to the exterior of an open container. Optionally, the fluid which perfuses the polymer-fluid matrix is the same fluid as that used to swell the polymer. Alternatively, the polymer may absorb a first fluid and the polymer-fluid matrix may be perfused by a second fluid.

The thermal properties of the polymer-fluid matrix can be adjusted by several means. Optionally, the polymer can be of varying molecular length or contain pendant groups, branching, co-polymers, and other factors to adjust the thermal properties of the polymer-fluid matrix. Optionally, the fluid can contain ions, salts, aqueous solvents, dissolved compounds, and other factors to adjust the thermal properties of the polymer-fluid matrix. For example, heat sensitive color dyes could be employed to provide an optical indicator for temperature changes. One of skill in the art would understand how various compounds and reagents alter the thermal properties of well-known fluids, for example elevation or depression of the boiling point for water. Additional means to adjust the thermal properties of the matrix include, but are not limited to, adjusting the ratio of co-polymers, adjusting the percentage of pendancy and/or branching, adjusting the ratio of multiple pendant groups, dissolving a mixture of components in the fluid, and the like.

Optionally, the device further contains a unit to measure the temperature of the polymer-fluid matrix. Tissue cooling devices and systems are typically positioned to contact the surface of the tissue (e.g., brain) and supply a cooled interface thereto. Inefficiencies in heat transfer results in a gradation of temperatures from the interface to points distal to the interface. The unit can optionally measure temperature at the surface and/or one or more depths within the polymer-fluid matrix. Assigning a depth of zero to the exterior surface of the polymer-fluid matrix, “depth” is determined to be the distance extending from a depth of zero inward to a point within the matrix. Depth can be in reference to the exterior surface of the matrix which interfaces with a cooling device. For instance, depth can refer to measurement of a point in the matrix which is about 10 mm directly below a portion of the matrix surface that interfaces with a cooling device. An embodiment of the present invention includes adjusting the temperature of the matrix to a determined temperature, subsequently cooling the surface of the matrix, and determining the temperature of the matrix at particular depths. The unit can optionally measure temperature at depths from about 0 to about 50 mm, from about 0 to about 40 mm, from about 0 to about 30 mm, from about 0 to about 20 mm, or from about 0 to about 15 mm. The unit can optionally measure temperature at depths from about 5 mm to about 15 mm. The unit can optionally measure temperature at more than one depth at approximately the same time or at approximately the same frequency. For instance, the unit can optionally measure temperature at depths of about 5 mm, about 10 mm, and about 15 mm at approximately the same time.

The unit to measure the temperature of the polymer-fluid matrix at one or more depths can be any device capable of accurately measuring the temperature of the matrix and/or perfusing fluid in the matrix. The unit can optionally be a thermocouple. The unit can have one or more means to measure temperature, e.g. one or more temperature sensing probes, prongs, or wires. For example, MRI-compliant temperature probes may be appropriate to accommodate procedures involving MRI. For embodiments in which temperature is measured at depths of about 5 mm, about 10 mm, and about 15 mm at approximately the same time, at least three single-probe units, a single probe unit and a double probe unit, or a triple-probe unit is required. More than one unit may be used in the device, and additional units may be positioned in separate locations within the device, e.g. near inlets and outlets to measure the temperature of inflowing and outflowing fluids. Commercially available units to measure the temperature of the matrix are available from National Instruments. For example, thermocouples: Omega K-type; DAQ system: NI cDAQ 9174; and module: NI 9211(×2). Optionally, the unit measures the temperature at a set frequency, for example between about 0.001 and about 1000 Hz, between about 0.01 and about 100 Hz, between about 0.1 and about 10 Hz, or between about 0.5 and about 5 Hz. Optionally, the unit measures the temperature at a frequency of about 1 Hz. Optionally, the unit is capable of measuring additional parameters such as pressure, flow rate, solute concentration, and the like.

As discussed, fluid flow dynamics are influenced in part by the properties of the interstitial spaces between matrix particles. Larger spaces can insulate the matrix against undesirable temperature and pressure fluctuations, among other benefits. However, larger spaces between matrix particles can result in microenvironments having slightly different properties, e.g. flow rate, pressure, and/or temperature. More accurate, repeatable temperature measurements are obtained from regions of the matrix containing smaller spaces between the matrix particles. In some embodiments, the matrix includes varying space sizes to mimic the larger and smaller vessels in human tissue. Optionally, the unit to measure the temperature of the polymer-fluid is positioned within the smallest particles of the polymer-fluid matrix. In some embodiments, the smallest particles are placed at or near the matrix-cooling device interface.

Tissue cooling devices and systems are typically applied to the outer surface of the tissue (such as brain tissue). The herein described matrix and perfusing fluid models the thermal properties of biological tissues or organs such as brain and/or blood. As with externally applied cooling to brain tissue, steeper and more significant decreases in temperature are observed near the matrix-cooling device interface than are observed deeper in the matrix and farther from the matrix-cooling device interface. For example, a matrix at physiological levels may experience a temperature decrease at 5 mm depth of about 8-10° C. in about 200 seconds when 2° C. cooling is applied to the matrix surface. Under similar circumstances but at a depth of 10 mm, a temperature decrease of only 6-7° C. may be observed, which may or may not reach a decrease of 8-10° C. Optionally, the thermal cooling property for at least a portion of the polymer-fluid matrix contained therein has a thermal property that deviates less than about 5° C., about 3° C., about 2° C., or about 1° C. from that of normal brain for a time that is substantially similar to the time used to cool a human brain. Table 1 below includes example data for human tissues as compared to water and SAP.

Tissue cooling techniques vary in duration, among other parameters. Limitations to cooling techniques include using cooling at no less than about −5° C., preferably no less than about 0° C., and more preferably at about 20 to about 25° C. Because the difference between cooling and physiological temperatures is relatively small, heat distribution and temperature stabilization is on the order of seconds to minutes. Because live tissue directly receives cooling, stronger temperature gradients using, e.g. dry ice, generally cannot be used as tissue may be irreversibly damaged. Thus, thermal cooling properties are best evaluated over time rather than as an end-result. In some embodiments, the time for assessing the thermal cooling properties of the polymer-fluid ranges from about 0.1 seconds to about 10 hours, from about 10 second to about 5 hours, or from about 200 seconds to several hours, e.g. 2-4 hours. In some embodiments, the time for assessing the thermal cooling properties of the polymer-fluid is up to about 2 hours, up to about 1 hour, up to about 30 minutes, up to about 10 minutes, or up to about 5 minutes.

Evaluation of a new technique, experimental method, or device, e.g. a brain cooling device, requires comparison to a standard and/or selected criteria. In the field of brain cooling devices and techniques, thermodynamic conditions e.g. thermal cooling can optionally be modeled by formula 1:

$$ρ {C}_{p , tis} ( T ) \frac{\partial T}{\partial t} = k ∇ T + {{ω}_{bl} ( ρ {C}_{p} )}_{bl} ( T - {T}_{bl} ) \qquad Formula 1$$

wherein ρ is the density, C_{p,tis} is the specific heat of the tissue, T is the temperature,

$$\frac{\partial T}{\partial t}$$

is the partial derivative of temperature with respect to time, k is the thermal conductivity of the tissue, ∇T is the gradient of the temperature in space, ω_{bl} is a blood perfusion term, (ρCₚ)_{bl} is the density of blood multiplied by the specific heat of blood, and (T−T_{bl}) is the difference in temperature between the system temperature and the blood temperature. Referred to as the Pennes bioheat equation, Formula 1 models the transient temperature response, which refers to the theoretical temperature of the matrix at a specific depth at a specific time after cooling is initiated. The calculation is based on the finite difference method, which refers to a temporal-spatial method to discretize a domain and solve differential equations. Formula 1 is derived from an energy balance on a control volume, with the inclusion of a perfusion term ω_{bl} defining the non-directional flow of blood in tissue parenchyma, which physiologically represents the thermal influence of blood circulation. The mathematical model utilizes a one-dimensional body of a material of specified properties, e.g. a polymer-fluid matrix, which experiences convection at both boundaries, and perfusion of some fluid throughout. By specifying the material properties, fluid properties, as well as the length of space and amount of time of the simulation, Formula 1 calculates how the temperature changes over time throughout the spatial domain.

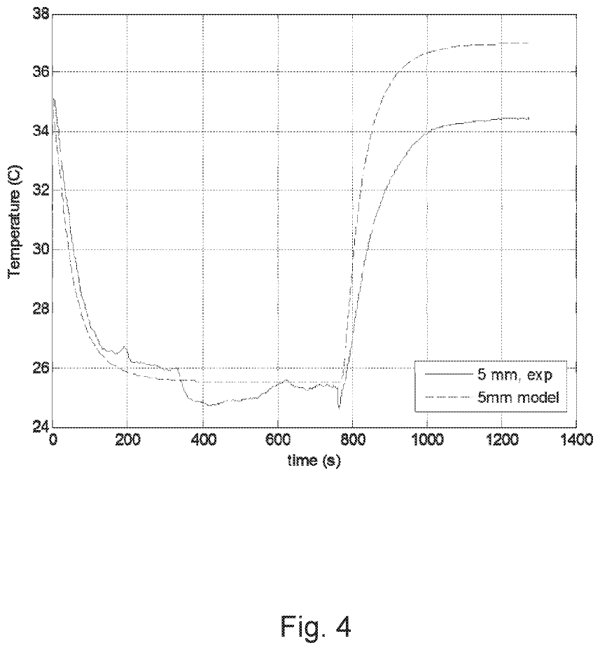

Changes in experimentally determined temperatures over time at a given depth in the matrix can be charted against theoretically modeled values under the same conditions derived from Formula 1. As an example, FIG. 4 is a graph comparing experimentally measured temperatures of the polymer-fluid matrix to the theoretical model at a frequency of 1 Hz and a depth of 5 mm after applying 2° C. cooling. FIG. 4 shows the device produces results that closely track the theoretical model, validating the device as effective for evaluating and/or monitoring therapeutic cooling of the brain. In some embodiments, the thermal cooling property deviates less than about 2° C., less than about 1.5° C., less than about 1° C., less than about 0.5° C., or less than about 0.2° C. from that of the modeled condition for at least about 600 seconds. In some embodiments, the thermal cooling property at a depth of about 5 mm deviates less than about 2° C., less than about 1.5° C., less than about 1° C., less than about 0.5° C., or less than about 0.2° C. from that of the modeled condition for at least about 600 seconds.

### Systems for Evaluating Tissue Cooling

In another embodiment, the invention comprises a system for evaluating tissue cooling (e.g., brain cooling) comprising: (a) a container comprising a polymer-fluid matrix having a thermal cooling property that is substantially similar to an organ or tissue such as human brain; and (b) a warm loop. In some embodiments, the warm loop contains a fluid having a temperature of about physiological temperature when passing through the inlet of the container. In some embodiments, the perfusion of flow ranges from about 30,000 to about 35,000 W/m³C. In some embodiments, flow rate within the warm loop ranges from about 2 to about 7 gph.

The system can include any of the herein described devices, e.g. a container comprising a polymer-fluid matrix having a thermal cooling property that is substantially similar to a tissue such as human brain. The device may be modified in numerous ways to accommodate features of the system. Non-limiting examples of modifications may include addition of or differential positioning of inlets and outlets to accommodate a warm loop(s), addition of units to measure inlet and outlet temperature of the warm loop, tubing to deliver fluid, etc.

The warm loop is optionally connected to the device, e.g. to the container. The warm loop optionally delivers and receives fluid to and from the device, e.g. to/from the container. The warm loop can be comprised of a system of tubes which loop fluid to and from the container and a warming device. The tubing can be any tubing capable of carrying fluids, e.g. plastic, rubber, or polyurethane tubing. Preferably, the tubing is made of chemically inert materials. In some embodiments, the tubing may be insulated. In some embodiments, the interior of the tubing may be coated to prevent leakage and/or avoid undesirable interactions between the tubing material and the circulating fluid. The tubing is optionally connected to an inlet(s) and/or outlet(s) on the container to deliver and receive fluid to/from the container. The tubing is optionally connected to an inlet(s) and/or outlet(s) on the warming device to deliver and receive fluid to/from the warming device. Commercial tubing is available from many suppliers, in a wide range of sizes. The warming device may be any device capable of increasing the temperature of a fluid. For instance, the warming device may be any standard bench-top water heater. Commercial warming devices are available from a range of suppliers, e.g. a tankless water heater from Eemax (e.g., PolyScience Model: LX Immersion Circulator, Model No. LXC-9A1 1B 120V/60 Hz. The warm loop may optionally contain a pump to circulate the fluid. The pump may be an additional component within the loop or provided in a commercial warming device.

The warm loop includes a fluid or a fluid containing dissolved gases. As the name implies, the warm loop comprises elevated temperatures, for instance in comparison to a cold loop. The warm loop optionally adjusts the temperature in the device, for instance by delivering warm fluid to the device. In some embodiments, the warm loop maintains the temperature in the device. As such, the warm loop may be the primary mediator of temperature within the container of the device. Thus, the system may optionally include an inlet for pumping fluid into the container and an outlet for removing fluid out of the container. In some embodiments, the warm loop contains a fluid passing through the inlet of the container having a temperature ranging from about 0° C. to about 50° C., from about 10° C. to about 45° C., from about 20° C. to about 43° C., from about 30° C. to about 40° C., from about 32° C. to about 38° C., or from about 35° C. to about 37° C. In some embodiments, the warm loop contains a fluid having a temperature of about physiological temperature when passing through the inlet of the container.

In some embodiments, the warm loop delivers a fluid to the container, wherein the fluid perfuses the polymer-fluid matrix. Such fluid perfusion can be a means to adjust or maintain the temperature within the container. A polymer swollen with fluid, e.g. a polymer comprising over 90% water, is thus susceptible to fluid-mediated temperature adjustment. Further, spacing between polymer particles in the matrix permits perfusion of the fluid between particles, thus providing uniform temperature adjustment and maintenance within the matrix of the container. This spatial arrangement mimics hemostatic blood circulation in vivo, e.g. in the brain. The perfusion of flow can be adjusted to model the level of perfusion of flow in capillaries in the tissue. Perfusion of flow is expressed in the equation perfusion=ω_{bl}c_{p,bl}, wherein ω_{bl} is blood perfusion in units of volumetric fluid flow per volume of tissue, and c_{p,bl} is the specific heat of blood. As the amount of perfusion increases, temperature uniformity and rapidity in which temperature can be adjusted increases. In some embodiments, the perfusion of flow ranges from about 1,000 to about 100,000 W/m³C (Watts per cubic meter per degree C.), from about 10,000 to about 75,000 W/m³C, from about 20,000 to about 50,000 W/m³C, from about 30,000 to about 35,000 W/m³C, or from about 32,000 to about 33,000 W/m³C.

Another parameter which affects the thermal properties within the matrix is the fluid flow rate. Faster flow rates mediate more rapid temperature adjustments. Because of this, faster flow rates are also less resistant to local temperature perturbations. Cerebral blood circulation has an average flow rate of about 4 gallons per hour (gph). Thus, the flow rate of fluid circulating in the warm loop may be adjusted to further modulate thermal conditions within the matrix. In some embodiments, the flow rate within the warm loop ranges from about 0 to about 10 gph, from about 1 to about 9 gph, from about 2 to about 8 gph, from about 2 to about 7 gph, from about 3 to about 6 gph, or from about 4 to about 5 gph. In some embodiments, the flow rate within the warm loop is about 4 gph.

In addition to the warm loop, the system optionally may further contain a cold loop. The cold loop is optionally connected to the device, e.g. to the container or to a second device positioned in the container. The cold loop optionally delivers and receives fluid to and from the device, e.g. to/from the container or to a second device positioned in the container. The cold loop can be comprised of a system of tubes which loop fluid to and from the container and a cooling device. The tubing can be the same, similar, or different as compared to the tubing of the warm loop. The tubing is optionally connected to an inlet(s) and/or an outlet(s) on a second device positioned within the container, e.g. a cooling device, to deliver and receive fluid to/from the second device. The tubing is optionally connected to an inlet(s) and/or outlet(s) on a third device, for example a source of cooling for the cold loop, to deliver and receive fluid to/from the warming device. The source of cooling for the cold loop can be, for example, an ice bath or a refrigerant device. The cooling device may be any device capable of locally decreasing the temperature of a contacted surfaces, e.g. the surface of the matrix. For instance, the cooling device may be any standard bench-top cooling block. Commercial cooling devices are available from a range of suppliers, e.g. an aluminum water-supplied cooling block from Koolance or 50 mm×50 mm blue pure copper base water cooling waterblock for CPU cooler manufactured by BQLZR. The cold loop may optionally contain a pump to circulate the fluid. The pump may be an additional component within the loop or provided in a commercial cooling device.

The cold loop includes a fluid or a fluid containing dissolved gases. As the name implies, the cold loop comprises reduced temperatures, for instance in comparison to a warm loop. The cold loop optionally adjusts the temperature in a cooling device, for instance by delivering cold fluid to the device. Thus, in some embodiments, fluid within the cold loop does not mix with fluid in the warm loop or in the container. In some embodiments, the cold loop maintains the temperature of the cooling device. In some embodiments, the cold loop contains a fluid having a temperature up to about 1° C., up to about 5° C., up to about 10° C., up to about 15° C., up to about 20° C., up to about 30° C., or up to about 50° C. lower than the temperature of the fluid in the warm loop. In some embodiments, the cold loop contains a fluid having a temperature less than physiological temperature. In some embodiments, the cold loop contains a fluid having a temperature ranging from about −5° C. to about 37° C., from about 0° C. to about 30° C., from about 1° C. to about 20° C., from about 2° C. to about 10° C., or from about 2° C. to about 5° C. In some embodiments, the cold loop contains a fluid having a temperature from about 0° C. to about 5° C., or of about 2° C.

The system may further comprise a fixture to which components of the system are affixed. The fixture may be used to fix components of the system in place, for instance to position the warm loop adjacent the container. As used herein, “fixture” refers to any device capable of positioning another object in a substantially stable position. Non-limiting examples of fixtures include any clamp, vice, scaffold, clip, wrapping element such as string or wire, magnet, adhesive surface, etc. However, tissue cooling measurements are sensitive to a number of factors. For instance, local variation in perfusion, fluid flow, particle size and spacing, etc. affect tissue cooling measurements and thus, the location on the matrix from which experiments proceed and data are recorded can influence results. As another example, the amount of pressure applied by, for instance a cooling device, on the matrix can alter local fluid and thermal dynamics and thus, the contact pressure applied when positioning a cooling device is of concern. As yet another example, the angle at which a device is positioned on the matrix creates similar concerns. Thus, in some embodiments, the fixture can be used for precision placement of system components. As an example, the fixture can be used to position the cold loop, or more specifically a cooling device of the cold loop, in a desired position within the container. In some embodiments, the fixture positions the cooling device on the polymer-fluid matrix at a repeatable height, contact pressure, and/or placement angle.

In some embodiments, the fixture comprises a sliding element and a guide element, positioned such that the sliding can slide along the guide element. In some embodiments, the sliding element further comprises an attaching element such that one or more devices may be attached to the sliding element. For example, a cooling device may be attached to the attaching element. In some embodiments, the sliding element further comprises a surface element attached to at least two base elements, the base elements capable of sliding. In such embodiments, the base elements can be attached to the surface element. In such embodiments, the surface element further comprises the attaching element. The surface element provides a surface to which the attaching element attaches. In some embodiments, the surface element and the at least two sliding elements slide together as a single unit, a sliding element. Upon sliding to a desired position along the guide, the sliding element is optionally capable of being fixed in that position along the guide. This represents sliding in one dimension of space. Optionally, each of the two or more base elements further comprise a base sliding element. The base sliding elements slide along the base elements in a direction substantially perpendicular to the direction the sliding element slides in. In such embodiments, the surface element is connected to the base sliding element. Optionally, the surface element is capable of sliding along the base elements in a direction substantially perpendicular to the direction the sliding element slides in. Upon sliding to a desired position along the base sliding elements, the surface element is optionally capable of being fixed in that position. This represents sliding in a second dimension of space substantially perpendicular to the first dimension of sliding. Optionally, the guide element is attached to a vertical element. Optionally, the guide element contains one or more guide sliding elements which slide along the vertical element. Optionally, the entire fixture is capable of sliding in a vertical direction by sliding the guide along the vertical element. Upon sliding to a desired position along the vertical element, the guide element is optionally capable of being fixed in that position. This represents sliding in a third dimension of space substantially perpendicular to the first dimension of sliding, and substantially perpendicular to the second dimension of sliding. As such, the fixture is capable of sliding motion in three-dimensional space, and further capable of being fixed in a position within three-dimensional space. Optionally, the fixture is made of solid material such as plastics, polymers, or metals and is highly stable against undesired tipping, bending, stretching, warping, sagging, or distorting. Preferably, the fixture comprises stainless steel components.

### Methods for Evaluating Tissue Cooling

In yet another embodiment, the invention comprises a method for evaluating a technique for cooling a tissue or organ such as brain, comprising (a) providing a device comprising a container that contains a polymer-fluid matrix having a thermal cooling property that is substantially similar to a human tissue or organ such as brain, and (b) perfusing the polymer-fluid matrix with a fluid. In some embodiments, perfusing the polymer-fluid matrix with a fluid maintains the temperature of a portion or all the polymer-fluid matrix at about physiological temperature. In some embodiments, the method further comprises measuring the temperature of the polymer-fluid matrix at a depth of up to about 20 mm. In some embodiments, the method further comprises cooling the polymer-fluid matrix with a cold loop. In some embodiments, the method further comprises comparing the measured temperatures to a modeled condition for thermal cooling.

The method can include any of the herein described devices, e.g. a container comprising a polymer-fluid matrix having a thermal cooling property that is substantially similar to a tissue or organ such as human brain. The device may be modified in numerous ways to accommodate features of the method. Non-limiting examples of modifications may include an open container to facilitate a step of placing a cooling device, inclusion of outlets, inlets, pumps, and tubing to facilitate the step of perfusing the matrix, etc.

The perfusing step may be facilitated by inlets and outlets on the container, which permit inflow and outflow of a fluid. The perfusing step may be accomplished by several methods, including continuous or intermittent flow of perfusing fluid. Optionally, the perfusing fluid may originate from one or more sources and terminate at one or more sinks, e.g. the perfusing fluid is not recycled. Alternatively, the perfusing fluid may be partially or completely recycled, e.g. within a fluid loop. In one embodiment, the fluid in the perfusing step is supplied by a warm loop, and thus the perfusing fluid adjusts or maintains the temperature of a portion or all the matrix. In some embodiments, perfusing the polymer-fluid matrix with a fluid maintains the temperature of a portion or all the polymer-fluid matrix at a temperature ranging from about 0° C. to about 50° C., from about 10° C. to about 45° C., from about 20° C. to about 43° C., from about 30° C. to about 40° C., from about 32° C. to about 38° C., or from about 35° C. to about 37° C. In some embodiments, perfusing the polymer-fluid matrix with a fluid maintains the temperature of a portion or all of the polymer-fluid matrix at about physiological temperature.

The method optionally further includes measuring the temperature of the container and/or the polymer-fluid matrix. Temperature may be measured at any locale within the container and/or the matrix. For instance, the temperature may be measured for inflowing and outflowing fluids, the surface of the matrix, and/or internal portions within the matrix. In some embodiments, the method optionally includes measuring the temperature of the polymer-fluid matrix at a particular depths in the matrix. For example, the temperature of the matrix may be measured at depths ranging from about 0 to about 50 mm, from about 0 to about 40 mm, from about 0 to about 30 mm, from about 0 to about 20 mm, or from about 0 to about 15 mm. In some embodiments, the temperature of the matrix may be measured at depths ranging from about 5 mm to about 15 mm, or at depths of about 5 mm, about 10 mm, and about 15 mm. The temperature can optionally be measured at more than one depth at approximately the same time or at approximately the same frequency. For instance, the temperature can optionally be measured at depths of about 5 mm, about 10 mm, and about 15 mm at approximately the same time. Further, the temperature can optionally be measured at a set frequency, for example between about 0.001 and about 1000 Hz, between about 0.01 and about 100 Hz, between about 0.1 and about 10 Hz, or between about 0.5 and about 5 Hz. Optionally, the temperature can be measured at a frequency of about 1 Hz. Optionally, the additional parameters such as pressure, flow rate, solute concentration, and the like may be measured along with temperature.

The method may further comprise cooling using a cold loop. In some embodiments, the cooling step cools the polymer-fluid matrix with a cold loop. In some embodiments, the cold loop comprises a cooling device cooled by a fluid in the cold loop. The method optionally may further comprise contacting the cold loop, particularly the cooling device of the cold loop, to the matrix. Cooling is supplied by the cooling device at the interface of the cooling device and the matrix. The effect of cooling may be observed, e.g. measured, at various positions, e.g. depths, within the matrix. Thus, the optional step of measuring the temperature in the matrix may proceed prior to, subsequent to, and/or concurrent with the cooling step, or any combination thereof. In some embodiments, the cooling step reduces the temperature of at least a portion of the polymer-fluid matrix to a range from below physiological temperature to about −5° C., from below physiological temperature to about 0° C., from 35° C. to about 2° C., from 35° C. to about 5° C., from 35° C. to about 20° C., or from 35° C. to about 25° C. In some embodiments, the cooling step reduces the temperature of at least a portion of the polymer-fluid matrix to a range from 30° C. to about 10° C., from 30° C. to about 20° C., or from 30° C. to about 25° C.

Because the means by which cooling is applied to biological tissue or to a polymer-fluid matrix, mechanisms to provide repeatable application of cooling are advantageous. Thus, the method may further comprise attaching a cooling device to a fixture. In such embodiments, the cooling device is securely attached to the fixture in a desired angle and without substantial interruption of the cold loop.

The method optionally further comprises positioning a cooling device on the matrix. As discussed, positioning of the cooling device can locally affect the fluid and thermal properties of the matrix. The cooling device may be carefully positioned on the matrix, for instance by hand of a technician. Alternatively and preferably, the cooling device may be affixed to a fixture and positioned by adjusting the fixture. In some embodiments, fixture positions the cooling device on the polymer-fluid matrix at a repeatable height, repeatable contact pressure, repeatable placement angle, or any combination thereof. Because cooling requires application of cooling to the matrix, the step of cooling using a cold loop can occur subsequent or concurrent with the step of positioning the cooling device.

Evaluation of tissue cooling devices (e.g., brain cooling devices) is facilitated by comparison to a standard or selected criteria. Optionally, the method further comprises comparing the measured temperatures to a modeled condition for thermal cooling defined by the following model of formula 1:

$$ρ {C}_{p , tis} ( T ) \frac{\partial T}{\partial t} = k ∇ T + {{ω}_{bl} ( ρ {C}_{p} )}_{bl} ( T - {T}_{bl} ) \qquad Formula 1$$

wherein ρ is the density, C_{p,tis} is the specific heat of the tissue, T is the temperature,

$$\frac{\partial T}{\partial t}$$

is the partial derivative of temperature with respect to time, k is the thermal conductivity of the tissue, ∇T is the gradient of the temperature in space, ω_{bl} is a blood perfusion term, (ρCₚ)_{bl} is the density of blood multiplied by the specific heat of blood, and (T−T_{bl}) is the difference in temperature between the system temperature and the blood temperature. Referred to as the Pennes bioheat equation, Formula 1 models the transient temperature response, which refers to the theoretical temperature of the matrix at a specific depth at a specific time after cooling is initiated. By specifying the material properties, fluid properties, as well as the length of space and amount of time of the simulation, Formula 1 calculates how the temperature changes over time throughout the spatial domain. Thus, experimentally determined temperatures at a particular depth may be compared to theoretical values calculated in Formula 1. Assuming all other parameters are either similar or differ in immaterial or unsubstantial ways, a brain cooling device, technique, and/or method which results in experimentally determined data that more closely resembles the theoretical values calculated in Formula 1 is a better performing device, technique, and/or method which results in experimentally determined data that less closely resembles the theoretical values calculated in Formula 1. In some embodiments, the thermal cooling property deviates less than about 2° C., less than about 1.5° C., less than about 1° C., less than about 0.5° C., or less than about 0.2° C. from that of the modeled condition for at least about 100 seconds. In some embodiments, the thermal cooling property deviates less than about 2° C., less than about 1.5° C., less than about 1° C., less than about 0.5° C., or less than about 0.2° C. from that of the modeled condition for at least about 300 seconds. In some embodiments, the thermal cooling property deviates less than about 2° C., less than about 1.5° C., less than about 1° C., less than about 0.5° C., or less than about 0.2° C. from that of the modeled condition for at least about 600 seconds. In some embodiments, the thermal cooling property deviates less than about 2° C., less than about 1.5° C., less than about 1° C., less than about 0.5° C., or less than about 0.2° C. from that of the modeled condition for at least about 1000 seconds. In some embodiments, the thermal cooling property at a depth of about 5 mm deviates less than about 2° C., less than about 1.5° C., less than about 1° C., less than about 0.5° C., or less than about 0.2° C. from that of the modeled condition for at least about 100 seconds. In some embodiments, the thermal cooling property at a depth of about 5 mm deviates less than about 2° C., less than about 1.5° C., less than about 1° C., less than about 0.5° C., or less than about 0.2° C. from that of the modeled condition for at least about 300 seconds. In some embodiments, the thermal cooling property at a depth of about 5 mm deviates less than about 2° C., less than about 1.5° C., less than about 1° C., less than about 0.5° C., or less than about 0.2° C. from that of the modeled condition for at least about 600 seconds. In some embodiments, the thermal cooling property at a depth of about 5 mm deviates less than about 2° C., less than about 1.5° C., less than about 1° C., less than about 0.5° C., or less than about 0.2° C. from that of the modeled condition for at least about 1000 seconds.

### DETAILED DESCRIPTION OF THE FIGURES

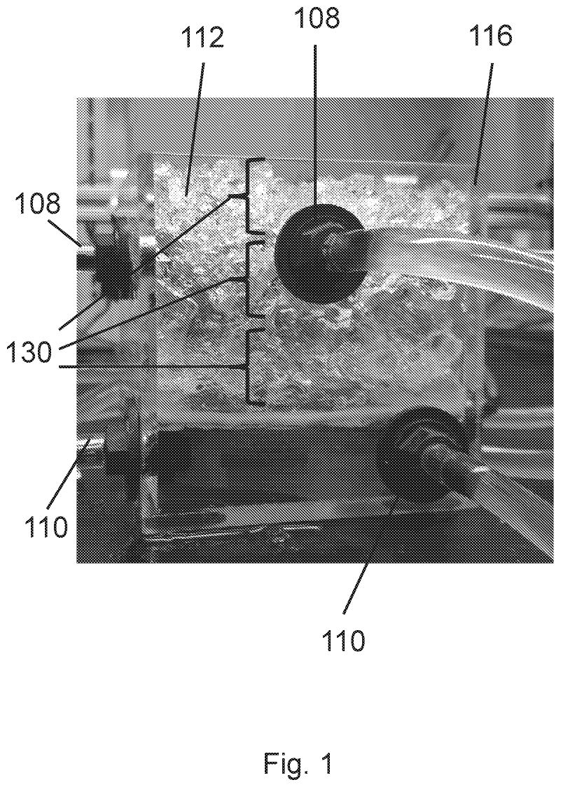

FIG. 1 is a photograph of an embodiment of an organ phantom device (e.g., brain phantom) for evaluating tissue cooling. In this embodiment four inlets 108 and four outlets 110 (two of each are visible here) move water or another liquid substance through the matrix of superabsorbent polymer (SAP) layers 112 held in a container 116. This inflow/outflow mimics perfusion of blood through the brain tissue. Thermocouples (not visible) are placed at varying depths in the polymer matrix to measure temperatures at different locations in the brain phantom. Different sized SAP polymers are used in layers 130 (as shown with brackets).

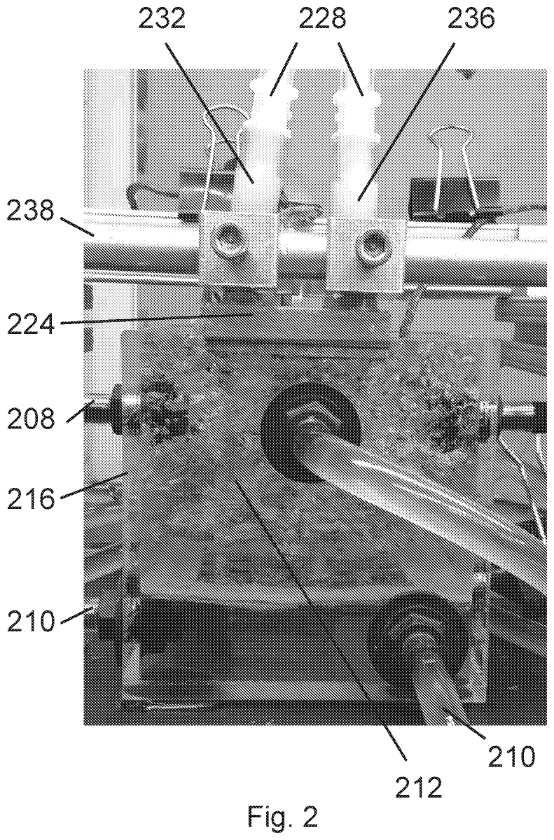

FIG. 2 is a photograph showing an embodiment of a method and system for using an organ phantom device (e.g., brain phantom) to evaluate tissue cooling capacity by a prototype copper CPU cooling system. Four inlets 208 and four outlets 210 (two of each are visible here) move water or another liquid substance through the matrix of superabsorbent polymer (SAP) layers 212 held in a container 216. The cooling device 224 is positioned at the top of the Thermocouples (not visible) are placed at varying depths in the SAP matrix 212 to measure temperatures at different depths in the brain phantom. A cooling system can be tested using the brain phantom, as shown here for a copper CPU cooling system. The cooling device 224 is connected to a closed-loop cycle of circulating water, and cooling is achieved due to a large reservoir of ice water (not shown) at the distal part of the loop. The inlet 232 conducts the cooled water into the cooling device 224 and the outlet 236 takes the water back to the ice water reservoir. Thermocouples 228 in the inlet and outlet measure temperature of the water as it moves through the inlet 232 and outlet 236. A fixture 238 maintained consistent placement of the cooling device 224 on the brain phantom SAP layers 212.

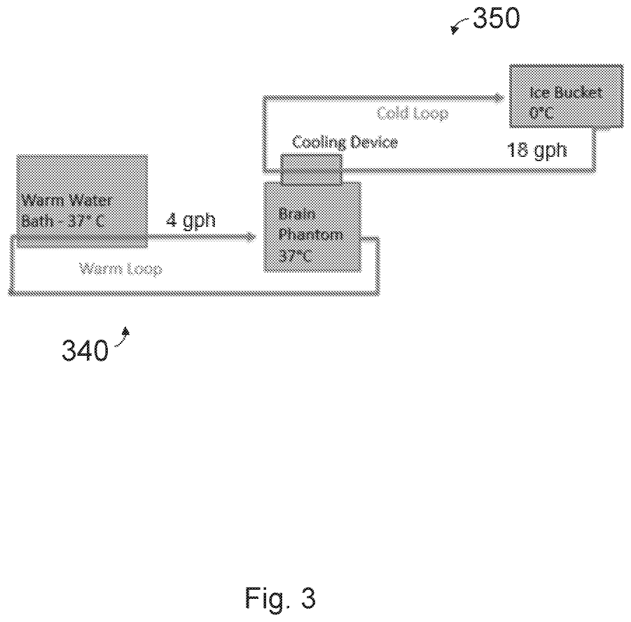

FIG. 3 is a schematic of the method and system for using an organ phantom device to evaluate tissue cooling capacity as shown in FIG. 2. Two closed-loop cycles of circulating water are established in this embodiment. The warm loop 340 represents the brain of a subject and mimics blood perfusion through the brain tissue. In this embodiment, water maintained at 37° C. is pumped at about 4 gallons per hour through the brain phantom, which is at the same temperature to start. The cold loop 350 is a fluid-cooled cooling device that acts as a heat sink, thereby cooling the brain phantom. An ice bucket maintains the lower temperature of the water in that loop, which is cycled at about 18 gallons per hour.

FIG. 4 shows a comparison of mathematically predicted cooling values (“model”) to data collected using a method and system for using a brain phantom device to evaluate brain cooling capacity as described above (“exp”). This graph represents the transient temperature response at 5 mm depth in the SAP matrix, where a thermocouple is embedded in the matrix to measure temperature. These data show that experimentally, the same steady-state value of 25.5° C. was achieved at 5 mm depth as predicted by the mathematical model.

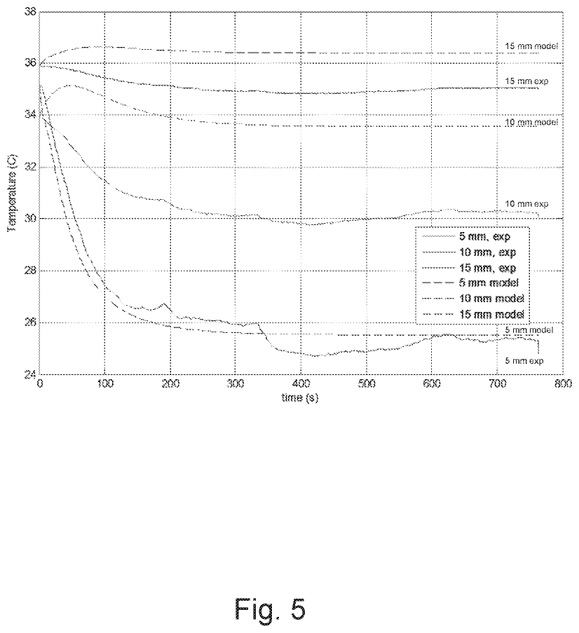

FIG. 5 shows comparisons similar to that in FIG. 4, but for all depths where data was recorded. Temperature measurements from thermocouples placed at 5, 10, and 15 mm depth in the brain phantom are shown over time. At the 5 mm depth, the brain phantom exhibited a response very close to that predicted mathematically, but at further depths the cooling surpasses predicted values. The results at 10 mm and 15 mm have comparable cooling patterns and reach steady-state temperatures within similar time periods, but measurements at those depths are much cooler than is predicted by the model.

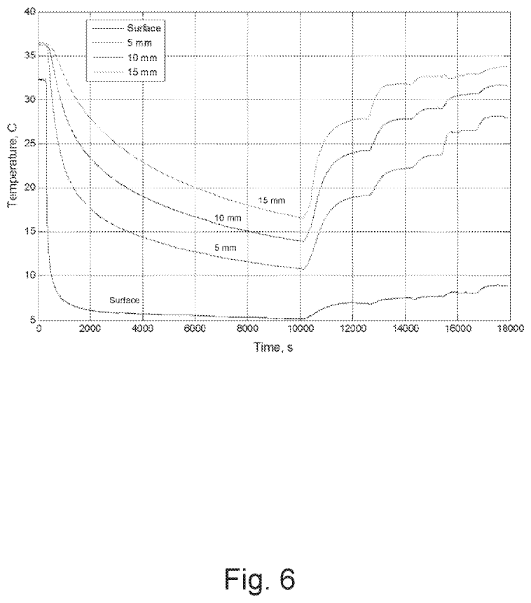

FIG. 6 shows the temperature profile effects over time at each of the different depths (surface, 5 mm, 10 mm, 15 mm) in the brain phantom with a perfusion rate of 4 gallons per hour. Other rates were tested, and 4 gallons per hour corresponded to the most physiologically accurate perfusion rate.

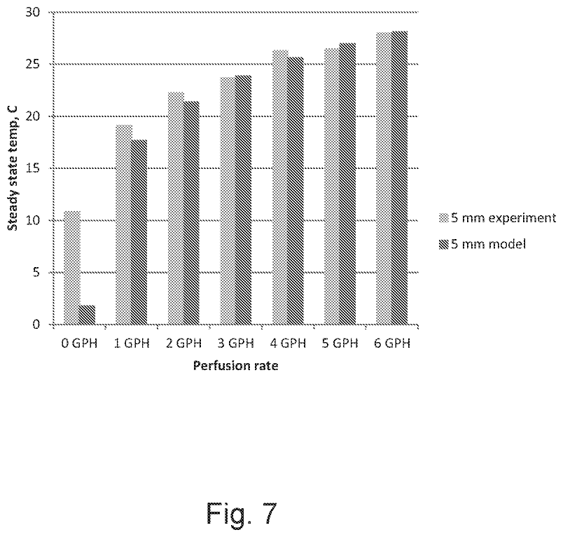

FIG. 7 shows comparisons of steady state temperatures achieved at 5 mm depth in the brain phantom for different perfusion rates, as predicted by mathematical model (“model”) and as experimentally observed (“experiment”). At 5 mm depth, the brain phantom demonstrates an appropriate qualitative response to increasing perfusion rate: as the perfusion rate of warm blood simulant increases so does the steady state temperature under the same cooling conditions.

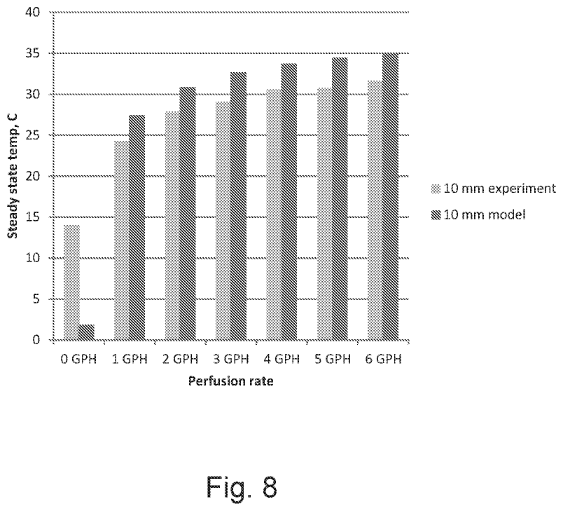

FIG. 8 shows the same comparisons as FIG. 7 but for 10 mm depth in the brain phantom. The trend of increasing steady state temperatures is observed, but the experimental observations were not as close to the values predicted by the model for 10 mm depth as for 5 mm.

### Example

The present disclosure can be understood in view of the following non-limiting examples, which are intended only for illustrative purposes. Any attribute or aspect of the following examples does not in any way limit the scope of the presently disclosed invention.

A physical laboratory model of a brain was developed and cooled in a series of tests to provide a prediction of the temperature response induced by various therapies. These results were compared against those of a mathematical model. The model can be used to predict the slight differences between the benchtop brain phantom and a human brain.

In these experiments, a fluid-cooled device was applied to the surface of a phantom brain device of the invention and monitoring the temperature drop at various depths below the surface. As shown in FIG. 3, to best simulate a cooling therapy, two closed-loop cycles of circulating water were established. The first loop was a fluid-cooled cooling device which acted as a heat sink, thereby applying cooling, designed to emulate therapeutic cooling, to the phantom brain. A pump was used to pump water from a reservoir of ice-water through a flow control valve-meter and into a cooling device. The second loop was designed to emulate warming of the brain via blood flow. The water used in this loop was kept warm through the use of a circulator and heat exchanger and then pumped through the brain phantom and then back through the loop in a cyclical manner. This brain phantom is equipped with several thermocouples which record the temperature at 1 Hz.

The two main attributes that a phantom must have in order to closely simulate a brain are thermal material properties similar to that of brain tissue and the ability to evenly distribute a heating fluid to mimic vascular circulation.

The brain phantom device was designed to closely emulate brain tissue. The material selected to closely represent brain tissue is a potassium neutralized super absorbent co-polymer (SAP) made of acrylic acid and acrylamide (AgSAP; M2 Polymer Technologies, Inc.). The polymer is provided as dry crystals, which when soaked in warm water for several hours, absorbs the surrounding water and swells to several hundred times the original volume. Because the hydrated polymer is approximately 99% water by volume, it has the thermal properties that are highly similar to water, and thus mimics the thermal properties of both brain and blood. Multiple SAP products were used in the phantom brains tested. including spheres, and various sizes of AgSAP, which are non-geometrical “chunks” of super absorbent polymer (SAP) material. The key thermal properties of each material and as used in the system are summarized in Table 1 below.

TABLE 1
Summary of material properties for blood, brain
tissue, and brain tissue simulant.

| | | Brain | | |
|---|---|---|---|---|
| | Blood | tissue | AgSAP | Water |
| Density, ρ | 1050 | 1040 | ~1000 | 1000 |
| (kg/m³) | | | | |
| Thermal | .52 | .5 | ~.63 | .63 |
| conductivity, | | | | |
| k (W/m * K) | | | | |
| Specific heat, | 3617 | 3560 | ~4178 | 4178 |
| Cₚ (J/kg * K) | | | | |

As core body temperature is maintained by heat which comes from cellular metabolism and this heat is distributed throughout the body via circulation of blood, to thermally model the brain or any living tissue, it is necessary to replicate the hemostatic effects of blood circulation. In targeting focal epilepsy, it is most relevant to model the cerebral cortex, where focal seizures primarily occur. This region is largely vascularized by capillaries and so when creating a brain phantom to mimic this region, it is very important to reproduce this widely distributed, even flow rather than targeting the modeling of one particular artery. This capillary flow was modeled by filling a small container with the SAP and introducing a flow of warm water, where the spaces between individual grains of the SAP represent the capillaries, and the flow trickles or leaches through.

The brain phantom SAP material was contained within a 4″×4″ acrylic box with an open top that was outfitted with 8 bulkhead fittings. For the prototype used in these experiments, there were 4 fittings near the bottom corners, through the vertical faces to serve as outlets for the water and 4 fittings near the top in the center of the vertical faces to serve as inlets for the water.

To assemble the phantom brain, the box was filled with about 1 inch of water. Then, a custom-designed filter screen was installed in the bottom of the box, parallel to the bottom face of the box and just below the water line. Then, about 96 grams of 1-mm SAP spheres were added to the box, which when distributed evenly on top of the filter screen is about enough to make two layers of spheres. Because of their relatively large size, and ability to hold their round shape, these spheres are put in this location up against the filter screen to help enable smooth flow of water down through the box, without the filter screen getting clogged by smaller particles. Additionally, 122 g of large grain AgSAP was then placed carefully in an even layer on top of the layer of spheres. Next, two custom-made tubes were installed into the inlet ports of the box. Each tube was made of soft PVC, measured approximately 3″ long and had many holes drilled in. Each tube was connected to two inlet ports, such that the water entering the container (i.e., the phantom brain) converges in the tubes, and then trickles out of the holes. This design was use to emulate a large artery branching into smaller vessels. The tube were of a material that could bend in a horizontal plane just above the top of the layer of large-grain SAP. Once the tubes were in place, about an equal weight amount (i.e., 115 g) of medium-grain SAP was added to the box in another neat layer. This mass was spread evenly all across the box so as to just barely cover the tops of the inlet tubes. This arrangement of different sized polymers allows for both proper blood flow and good spatial resolution. FIG. 1 depicts the phantom arrangement having the different layers indicated as element 130.

Once the box was appropriately layered with SAP and outfitted with inlet tubes, the box was outfitted with thermocouples to monitor the temperature in the brain phantom in several key locations. One key aspect of the device is the thermocouple fixture which holds the three thermocouples that monitor the internal temperature of the brain phantom at the three depths of 5, 10, and 15 mm. This fixture was held in place by clamping the thermocouple wires to the a fixed structure, such that the thermocouple fixture was held in the center of the box, right in between the two inlet tubes, at a level angle. Other thermocouples can be positioned accordingly as needed.

Once the internal thermocouples were in place, a small amount of medium-grain SAP (about 10-20 g for a 4″ by 4″ by 4″ box) was chopped into very fine particles and placed directly around the internal thermocouples. Placing this small-grain AgSAP directly around the thermocouples provided relatively high resolution and minimized the impact of individual grain placement on local flow paths in the area where the temperature is being measured.

Once the small-grain SAP was added to the brain phantom, two more thermocouples were added. One thermocouple measured the temperature of the warm fluid just as it entered the box, and was inserted into one of the holes near the edge of the box in one of the inlet tubes. It was found to be desirable to have the thermocouple protrude into the bulkhead space. Monitoring this temperature is important to ensure that the perfusate entering the box stays steady at body temperature, to mimic blood flow. The second thermocouple was used to measure the temperature of the surface of the brain phantom, and when the cooling device is placed, can also measure the surface of the cooling device, serving as an interface probe.

A commercially available water-cooled CPU cooling block was used in the test setup to emulate a cooling device. This cooling device was held in place by a fixture which ensured consistent placement during each test. The fixture maintained consistent placement by controlling six degrees of freedom of the cooling device: a consistent height, contact pressure, left-right position, and placement angle such that the conducting surface of the cooling device rested lightly and flat against the top surface of the brain phantom (i.e., the polymer mix) on every placement. This is shown as element 238 in FIG. 2.

The cooling device was also equipped with two thermocouples, one of which measured the temperature of the cold fluid coming into the cold device and the other of which measured the temperature of the fluid leaving the cold device. The first temperature ensured a sufficiently cold fluid is being used as a coolant, and the difference in the two temperatures helps to indicate the performance of the cooling device as a heat sink.

One test used to verify the accuracy of the brain phantom as a predictor of cooling device performance on an actual brain analyzed the effect of perfusion rate on the ability of the device to cool the phantom. In order to closely mimic the physiology of the brain, warm water must not only be evenly distributed, but the flow rate must be appropriately scaled and controlled. Scaling cerebral blood flow by volume to the brain phantom used in these experiments utilized a perfusion rate of about 4 GPH to emulate human blood flow. Variation of flow rate of blood in the brain (or warm water in the test loop) has one of the largest effects on cooling of any test parameter and so may need to be varied as necessary to be consistent with the physiological state of the brain under therapeutic or surgical situations.

To test the cooling of the phantom brain, the pump which circulates the warm perfusate was turned on, allowing the brain phantom to warm up to core body temperature. Meanwhile, the cold pump was turned on as well, circulating the ice water and allowing the cooling device to reach a very cold steady state. Once each component had reached steady state, the device was placed in the fixture on top of the phantom, and the warm loop was turned off to allow the brain phantom to cool without perfusion.

When the internal temperatures of the brain phantom reached steady state, the next phase of the test was initiated. The warm pump was turned on and the control valve tuned to 1 GPH to warm the brain phantom by the introduction of the warm water, and allowed to reach steady state. Once steady state was achieved, the valve was opened to run at 2 GPH. This process was repeated at 1 GPH increments until steady state was achieved at 6 GPH. This test procedure gave the temperature profile at a large range of perfusion rates, which includes 4 GPH, which is estimated to be the most physiologically accurate perfusion rate (FIG. 6).

The test conditions can also be modeled mathematically to verify the set-up used for the brain phantom. Using a code developed by Gayzik et. al., J. Biomech. Eng., 128(4): p. 505-15 (2006), incorporated herein by reference in its entirety, the transient temperature response can be calculated using the finite difference method. The code is based upon the Pennes bioheat equation, shown in Equation 1 below:

$$ρ {C}_{p , tis} ( T ) \frac{\partial T}{\partial t} = k ∇ T + {{ω}_{bl} ( ρ {C}_{p} )}_{bl} ( T - {T}_{bl} ) \qquad [ 1 ]$$

The Pennes bioheat equation is derived from an energy balance on a control volume, with the inclusion of a perfusion term, which physiologically represents the thermal influence of blood circulation. The mathematical model utilizes a one-dimensional body of a material of specified properties, which experiences convection at both boundaries, and perfusion of some fluid throughout. By specifying the material properties, fluid properties, as well as the length of space and amount of time of the simulation, the finite difference code calculates how the temperature changes over time throughout the spatial domain.

The experimental results at a depth of 5 mm are shown in FIG. 7, as compared to the results predicted by the mathematical model. It is evident that the brain phantom shows an appropriate qualitative response to increasing perfusion rate: as the perfusion rate of warm blood simulant increases so does the steady state temperature under the same cooling conditions.

It was found that he benchtop phantom shows good agreement with the mathematical model. Additionally, by altering the model parameters from the properties of water (or SAP) to those of brain tissue, a prediction can be made as to how the brain will react under cooling. The experimental results show good agreement with the model of the brain as well, which builds great confidence in the ability of the benchtop brain phantom to predict how an actual, living human brain will react under similar cooling conditions. Specifically in the case of focal epilepsy, the 5 mm depth is important because early studies suggest that cooling the tissue at this depth by 12 C is the threshold is key to treating focal seizures, as discussed in Fujii et. al., Neurol. Med. Chir. (Tokyo), 50(9): p. 839-44 (2010), incorporated herein by reference in its entirety. Agreement of the experiment with the model is limited in the case of no perfusion (0 GPH) because of the time necessary to cool fully. Instead, a working definition of steady state was employed throughout the test, which said that if the temperature does not change more than 0.05 C in 2:00 minutes, the temperature has reached steady state. With a sufficient amount of ice and time, the temperature of the brain phantom being cooled without perfusion would converge upon the temperature of the cold water entering the device, as predicted by the mathematical model.

Temperature results from the 10 mm depth were also plotted against the model. The experimental results at the 10 and 15 mm depths (FIG. 8) also showed the trend of increasing steady state temperatures with increasing perfusion rate, however they showed less agreement with the results of the model at these depths.

While the preferred embodiments of the invention have been illustrated and described, it will be appreciated that various changes can be made therein without departing from the spirit and scope of the invention. All printed patents and publications referred to in this application are hereby incorporated herein in their entirety by this reference.

1. A device for evaluating tissue or organ cooling, comprising a container that contains a polymer-fluid matrix having a thermal cooling property that is substantially similar to a blood perfused human tissue or organ.

2. The device of claim 1, having an inlet for pumping fluid into the container and an outlet for removing fluid out of the container.

3. The device of claim 1, wherein the fluid perfuses the polymer-fluid matrix.

4. The device of claim 2, wherein the fluid pumped in and out of the container is water.

5. The device of claim 2, wherein the polymer is a super absorbent polymer that, when hydrated, has thermal properties similar to water.

6. The device of claim 2, wherein the polymer comprises two or more sizes of particles arranged in layers.

7. The device of claim 2, wherein the polymer is a co-polymer of acrylic acid and acrylamide.

8. The device of claim 2, wherein the polymer-fluid matrix has a density from about 950 to about 1,100 kg/m³.

9. The device of claim 2, wherein the polymer-fluid matrix has a specific heat from about 3400 to about 4250 J/kg-C.

10. The device of claim 2, further comprising a unit to measure the temperature of the polymer-fluid matrix at one or more depths.

11. The device of claim 10, wherein the unit to measure the temperature is a thermocouple.

12. The device of claim 10, wherein the unit to measure the temperature of the polymer-fluid is positioned within the smallest particles of the polymer-fluid matrix.

13. The device of claim 2, wherein the thermal cooling property for at least a portion of the polymer-fluid matrix contained therein has a thermal property that deviates less than about 2° C. from that of normal human tissue or organ for a time that is substantially similar to the time used to cool a human tissue or organ, such as a human brain.

14. The device of claim 2, wherein a time for assessing the thermal cooling properties of the polymer-fluid ranges from about 200 seconds to several hours.

15. The device of claim 2, wherein a modeled condition for thermal cooling is defined by the following model:
$$ρ {C}_{p , tis} ( T ) \frac{\partial T}{\partial t} = k ∇ T + {{ω}_{bl} ( ρ {C}_{p} )}_{bl} ( T - {T}_{bl} )$$

16. The device of claim 15, wherein the thermal cooling property at a depth of about 5 mm deviates less than about 0.5° C. from that of the modeled condition for at least about 600 seconds.

17. A system for evaluating tissue or organ cooling comprising: (a) a container comprising a polymer-fluid matrix having a thermal cooling property that is substantially similar to a human tissue or organ; and (b) a warm loop.

18. The system of claim 17, wherein the warm loop contains a fluid having a temperature of about physiological temperature.

19. The system of claim 17, wherein the perfusion of flow ranges from about 30,000 to about 35,000 W/m³C.

20. The system of claim 17, wherein the flow rate within the warm loop ranges from about 2 to about 7 gph.

21. The system of any of claims 17-20, wherein the flow rate within the warm loop is about 4 gph.

22. The system of any of claims 17-21, having an inlet for pumping fluid into the container and an outlet for removing fluid out of the container.

23. The system of any of claims 17-22, wherein the fluid permeates the polymer-fluid matrix.

24. The system of any of claims 17-23, wherein the fluid pumped in and out of the container is water.

25. The system of any of claims 17-24, wherein the polymer is a super absorbent polymer that, when hydrated, has thermal properties similar to water.

26. The system of any of claims 17-25, wherein the polymer comprises two or more sizes of particles arranged in layers.

27. The system of any of claims 17-26, wherein the polymer is a co-polymer of acrylic acid and acrylamide.

28. The system of any of claims 17-27, wherein the polymer-fluid matrix has a density from about 950 to about 1,100 kg/m³.

29. The system of any of claims 17-28, wherein the polymer-fluid matrix has a specific heat from about 3400 to about 4250 J/kgC.

30. The system of any of claims 17-29, further comprising a unit to measure the temperature of the polymer-fluid at one or more depths in the matrix.

31. The system of claim 30, wherein the unit to measure the temperature is a thermocouple.

32. The system of claim 30 or claim 31, wherein the unit to measure the temperature of the polymer-fluid is positioned within the smallest particles of the polymer-fluid matrix.

33. The system of any of claims 17-32, wherein the thermal cooling property for at least a portion of the polymer-fluid matrix contained therein has a thermal property that deviates less than about 2° C. from that of normal tissue, organ, or brain for a time that is substantially similar to the time used to cool a human tissue, organ, or brain.

34. The system of any of claims 17-33, wherein a time for assessing the thermal cooling properties of the polymer-fluid ranges from about 200 seconds to several hours.

35. The system of any of claims 17-34, wherein a modeled condition for thermal cooling is defined by the following model:
$$ρ {C}_{p , tis} ( T ) \frac{\partial T}{\partial t} = k ∇ T + {{ω}_{bl} ( ρ {C}_{p} )}_{bl} ( T - {T}_{bl} )$$

36. The system of claim 35, wherein the thermal cooling property at a depth of about 5 mm deviates less than about 0.5° C. from that of the modeled condition for at least about 600 seconds.

37. The system of any of claims 17-36, further comprising a cold loop.

38. The system of claim 37, wherein the cold loop comprises a fluid having a temperature less than about physiological temperature for cooling the polymer-fluid matrix.

39. The system of claim 37 or claim 38, further comprising a cooling device cooled by the fluid in the cold loop.

40. A method for evaluating a technique for cooling a tissue or organ comprising (a) providing a device comprising a container that contains a polymer-fluid matrix having a thermal cooling property that is substantially similar to a human tissue or organ, and (b) perfusing the polymer-fluid matrix with a fluid.

41. The method of claim 40, wherein the polymer is a super absorbent polymer that, when hydrated, has thermal properties similar to water.

42. The method of claim 40 or claim 41, wherein the fluid in the perfusing step is supplied by a warm loop.

43. The method of any of claims 40-42, wherein the step of perfusing the polymer-fluid matrix with a fluid maintains the temperature of a portion or all of the polymer-fluid matrix at about physiological temperature.

44. The method of any of claims 40-43, further comprising measuring the temperature of the polymer-fluid matrix at a depth ranging from about 0 to about 20 mm.

45. The method of any of claims 40-44, further comprising measuring the temperature of the polymer-fluid matrix at a depth ranging from about 5 mm to about 15 mm.

46. The method of any of claims 40-45, further comprising cooling the polymer-fluid matrix with a cold loop.

47. The method of claim 46, wherein the cold loop comprises a cooling device cooled by a fluid in the cold loop.

48. The method of claim 46 or claim 47, wherein the cooling device is attached to a fixture.

49. The method of claim 48, wherein the fixture positions the cooling device on the polymer-fluid matrix at a repeatable height, repeatable contact pressure, repeatable placement angle, or any combination thereof.

50. The method of any of claims 46-49, wherein the cooling step reduces the temperature of at least a portion of the polymer-fluid matrix to a range from below physiological temperature to about 0° C.

51. The method of any of claims 46-50, wherein the cooling step reduces the temperature of at least a portion of the polymer-fluid matrix to a range from about 35° C. to about 20° C.

52. The method of any of claims 40-51, further comprising comparing the measured temperatures to a modeled condition for thermal cooling defined by the following model:
$$ρ {C}_{p , tis} ( T ) \frac{\partial T}{\partial t} = k ∇ T + {{ω}_{bl} ( ρ {C}_{p} )}_{bl} ( T - {T}_{bl} )$$

53. The method of claim 52, wherein the thermal cooling property at a depth of about 5 mm deviates less than about 0.5° C. from that of the modeled condition for at least about 600 seconds.

54. The device of claim 1, wherein the polymer-fluid matrix has a thermal cooling property that is substantially similar to human brain tissue.

55. The system of claim 17, wherein the polymer-fluid matrix has a thermal cooling property that is substantially similar to human brain tissue.

56. The method of claim 40, wherein the method is used to evaluate a technique for cooling a human brain, and wherein the polymer-fluid matrix has a thermal cooling property that is substantially similar to human brain tissue.
